(19) United States
(12) Patent Application Publication
(10) Pub. No.: US 2006/0183122 A1
(43) Pub. Date: Aug. 17, 2006

(54) Fanc gene mutations in cancer

(76) Inventors: Scott E Kern, Hunt Valley, MD (US); Michael S Van Der Heijden, Amsterdam (NL)

Correspondence Address: EDWARDS & ANGELL, LLP, P.O. BOX 55874, BOSTON, MA, 02205, US

(21) Appl. No.: 10/540,904

(22) PCT Filed: Dec. 26, 2003
(86) PCT No.: PCT/US03/41127

§ 371 Date: Mar. 15, 2006

Related U.S. Application Data
(60) Provisional application No. 60/436,763, filed on Dec. 27, 2002.

(51) Int. Cl. C12Q 1/68

(52) U.S. Cl. 435/6

(57) ABSTRACT

The present invention relates to methods of determining if a patient has cancer or is at increased risk of developing cancer, particularly pancreatic cancer, the method comprising testing a FANC gene for the presence of a cancer-associated coding change, wherein said presence of one or more cancer-associated coding changes is indicative of cancer or an increased risk of cancer in said patient. The invention further relates to methods of treating a patient having cancer, particularly pancreatic cancer, who has one or more cancer-associated coding changes in the FANC genes comprising the step of administering a therapeutically effective amount of a chemotherapeutic cross-linking agent.

### RELATED APPLICATIONS

This application claims the benefit of U.S. Provisional Application Ser. No. 60/436,763, filed Dec. 27, 2002, the entire contents of which is incorporated herein by this reference.

### BACKGROUND OF THE INVENTION

Fanconi anemia (FA) is a rare and usually fatal human disorder, characterized by congenital bone deformities, progressive bone marrow failure and a predisposition to hematological malignancy (especially acute myelogenous leukemia) and squamous cell carcinoma of the head and neck, anogenital region, skin and other organs (Kutler, D. I. et al. A 20-year perspective on the International Fanconi Anemia Registry (IFAR). Blood 101, 1249-56; 2003). Studies have shown that FA is a recessive autosomal disorder. That is, it is an inherited disease which results from the presence of a mutated gene in both parents. Briefly, a gene which, when mutated, gives rise to FA in an individual may be referred to as an FA gene. Human cells are diploid, meaning that each cell has two copies of each chromosome and therefore two copies of each gene including each FA gene, one contributed from each parent. The recessive nature of the FA disorder means that both copies of a particular FA gene must be mutated in order for an individual to exhibit symptoms. Thus, it is assumed that FA sufferers carry one (or more) mutation(s) in both copies of a particular FA gene. A non-mutated, normal version of this gene encodes a protein that plays a role in a particular biochemical pathway of the cell. The normal protein is therefore required for overall normal cell function. The mutated FA gene encodes either a defective protein or no protein at all, and so the specific biochemical pathway for which the portion is required is changed, and thereby normal cell function is disrupted. Individuals who have one copy of an FA gene which is “normal” and one copy which is mutated do not exhibit FA symptoms but rather, are FA carriers. FA carriers may also be described as FA heterozygotes. Thus presumably, FA heterozygotes do not manifest clinical FA symptoms because they have one normal copy and one mutant copy of a particular FA gene, and that the protein produced by the one normal gene is sufficient for normal cell function (or at least sufficiently normal cell function so that no overt clinical abnormalities are presented). The offspring of two FA carriers who carry mutations in the same FA gene have a 25 percent chance of inheriting the FA disease and a 50 percent chance of being FA carriers themselves.

FA cells display spontaneous chromosome breakage, greatly enhanced by DNA-interstrand crosslinking agents such as mitomycin C (MMC) and diepoxybutane. Seven FA genes have been cloned so far, mutations in FANCA (65%), FANCC (15%) and FANCG (10%) account for the majority of cases (D'Andrea, A. D. & Grompe, M. The Fanconi anaemia/BRCA pathway. Nat Rev Cancer 3, 23-34, 2003; Joenje, H. & Patel, K. J. The emerging genetic and molecular basis of Fanconi anaemia. Nat Rev Genet 2, 446-57, 2001). The function of the FA pathway remains to be fully elucidated, but seems to be required for an adequate reponse to DNA damage as caused by these agents. BRCA2 mutations have been shown to be responsible for a subset of FA patients: complementation group D1 and perhaps B (Howlett, N. G. et al. Biallelic inactivation of BRCA2 in Fanconi anemia. Science 297, 606-9, 2002). Mutations in these patients affected both alleles and included at least one hypomorphic mutation per patient, in which some residual function may have remained. Complementation of a FANCD1 cell line with wild-type BRCA2 corrected the cytogenetically measured MMC-hypersensitivity. Herein, the proximal Fanconi pathway and BRCA2 are collectively referred to as the Fanconi pathway.

Pancreatic cancers harbor the highest percentages of BRCA2 mutations, present in 7% of sporadic pancreatic cancers (all accompanied by loss of the wild-type allele), 12% of familial pancreatic cancer and 17% of families with a strong history of the disease (Goggins, M. et al. Germline BRCA2 gene mutations in patients with apparently sporadic pancreatic carcinomas. Cancer Res 56, 5360-4, 1996; Murphy, K. M. et al. Evaluation of candidate genes MAP2K4, MADH4, ACVR1B, and BRCA2 in familial pancreatic cancer: deleterious BRCA2 mutations in 17%. Cancer Res 62, 3789-93, 2002; Figer, A. et al. The rate of the 6174delT founder Jewish mutation in BRCA2 in patients with non-colonic gastrointestinal tract tumours in Israel. Br J Cancer 84, 478-81, 2001; Hahn, S. A. et al. BRCA2 germline mutations in familial pancreatic carcinoma. J Natl Cancer Inst 95, 214-21, 2003). Pancreatic cancer, diagnosed in over 30,000 people in the United States yearly, is one of the most aggressive forms of cancer, leading to death in an overwhelming majority of patients within a few years despite surgery and/or chemotherapeutic treatment Several lines of evidence suggest the use of combinations of chemotherapy containing MMC and other crosslinking agents to be beneficial for pancreatic cancer patients. Although a significant increase in survival is usually not found, occasional complete and long-term remissions are reported (Sadoff, L. & Latino, F. Complete clinical remission in a patient with advanced pancreatic cancer using mitomycin C-based chemotherapy: the role of adjunctive heparin. Am J Clin Oncol 22, 187-90, 1999; Takada, T. et al. Prospective randomized trial of 5-fluorouracil, doxorubicin, and mitomycin C for non-resectable pancreatic and biliary carcinoma: multicenter randomized trial. Hepatogastroenterology 45, 2020-6, 1998; Todd, K. E., Gloor, B., Lane, J. S., Isacoff, W. H. & Reber, H. A. Resection of locally advanced pancreatic cancer after downstaging with continuous-infusion 5-fluorouracil, mitomycin-C, leucovorin, and dipyridamole. J Gastrointest Surg 2, 159-66, 1998). These reports have not incorporated the genetic testing of these patients, but a gene defect in BRCA2, FANCC, FANCG or another gene in the FA-pathway could in theory cause a therapeutically useful hypersensitivity, providing an “Achilles' heel” in a subset of pancreatic cancers. Perhaps the first link between (pancreatic) cancer and FA was observed as early as 1976 in a Scottish family: a consanguineous pedigree was described in which one person had FA, and obligate mutation carriers displayed multiple occurrences of pancreatic and other cancers (Hill, R. D. Familial cancer on a Scottish island. Br Med J 2, 401-2, 1976). Approximately seven percent of sporadic pancreatic cancers carry mutations in BRCA2, accompanied by loss of heterozygosity (LOH) (Goggins, M. et al. Germline BRCA2 gene mutations in patients with apparently sporadic pancreatic carcinomas. Cancer Res 56, 5360-4, 1996). The pancreatic cancer cell line CAPAN1 is derived from such a tumor.

As mentioned, FA cells have an increased sensitivity to crosslinking agents, especially MMC. The existence of FA-proficient hosts harboring pancreatic cancers that are defective in the FA pathway could have important implications for clinical treatment: the tumor could be hypersensitive to crosslinking agents, whereas the patient would not. Several studies have reported long-term remissions in pancreatic cancer in response to MMC, although the link with FA defects has never been evaluated clinically (Sadoff, L. & Latino, F. Complete clinical remission in a patient with advanced pancreatic cancer using mitomycin C-based chemotherapy: the role of adjunctive heparin. Am J Clin Oncol 22, 187-90, 1999; Takada, T. et al. Prospective randomized trial of 5-fluorouracil, doxorubicin, and mitomycin C for non-resectable pancreatic and biliary carcinoma: multicenter randomized trial. Hepatogastroenterology 45, 2020-6, 1998; Todd, K. E., Gloor, B., Lane, J. S., Isacoff, W. H. & Reber, H. A. Resection of locally advanced pancreatic cancer after downstaging with continuous-infusion 5-fluorouracil, mitomycin-C, leucovorin, and dipyridamole. J Gastrointest Surg 2, 159-66, 1998). The BRCA2-defective cell line CAPAN1 has been shown to be hypersensitive to ionizing radiation (IR) and some chemotherapeutics (Moynahan, M. E., Cui, T. Y. & Jasin, M. Homology-directed dna repair, mitomycin-c resistance, and chromosome stability is restored with correction of a Brca1 mutation. Cancer Res 61,4842-50,2001; Abbott, D. W., Freeman, M. L. & Holt, J. T. Double-strand break repair deficiency and radiation sensitivity in BRCA2 mutant cancer cells. J Natl Cancer Inst 90, 978-85, 1998; Chen, P. L. et al. The BRC repeats in BRCA2 are critical for RAD51 binding and resistance to methyl methanesulfonate treatment. Proc Natl Acad Sci USA 95, 5287-92, 1998).

In recent years, the emerging field of targeted chemotherapeutics, in particular the targeting of specific genetic defects in cancer, has received much attention. Practiced examples of such therapies are not often encountered. Defects in the FA-pathway may provide a vulnerable target for therapeutics (Moynahan, M. E., Pierce, A. J. & Jasin, M. BRCA2 is required for homology-directed repair of chromosomal breaks. Mol Cell 7, 263-72, 2001), specifically using the interstrand DNA-crosslinking agents. The hypersensitivity of cells taken from FA patients to crosslinking agents and to ionizing radiation already suggests this utility (Sasaki, M. S. & Tonomura, A. A high susceptibility of Fanconi's anemia to chromosome breakage by DNA cross-linking agents. Cancer Res 33, 1829-36, 1973; Auerbach, A. D. & Wolman, S. R. Susceptibility of Fanconi's anaemia fibroblasts to chromosome damage by carcinogens. Nature 261, 494-6, 1976). Although tumors that develop in FA patients cannot easily be treated with these therapies due to toxicity, FA-defective tumors in individuals who carry no mutation or only one (recessive) mutation in FA genes may offer a highly augmented therapeutic response to crosslinking agents, fortuitously with little anticipated toxicity to the patient (Tutt, A. et al. Mutation in Brca2 stimulates error-prone homology-directed repair of DNA double-strand breaks occurring between repeated sequences. Embo J 20, 4704-16, 2001; Kern, S. E., Hruban, R H., Hidalgo, M. & Yeo, C. J. An introduction to pancreatic adenocarcinoma genetics, pathology and therapy. Cancer Biol Ther 1, 607-13, 2002).

An early detection of defects in the FA pathway in pancreatic cancer could perhaps lead to a better treatment for some patients and a better assessment of risk for family members. In addition, identification of patients with defects in the FA pathway will potentially allow prophylactic and therapeutic methods of treating cancer with targeted chemotherapeutic agents.

### SUMMARY OF THE INVENTION

The present invention is based on the discovery that patients with one or more coding changes in FANC genes, resulting in mutations, are at increased risk of developing cancer, including pancreatic cancer.

The present invention pertains to methods of determining if a patient has cancer or is at increased risk of developing cancer, particularly pancreatic cancer, the method comprising testing a FANC gene for the presence of a cancer-associated coding change, wherein said presence of one or more cancer-associated coding changes is indicative of cancer or an increased risk of developing cancer in said patient.

In one embodiment, the cancer-associated coding change in the FANC gene is selected from the group consisting of: mutations of the FANCA gene listed in Table 2, mutations of the FANCC gene listed in Table 3, mutations of the FANCD2 gene listed in Table 4, mutations of the FANCE gene listed in Table 5, mutations of the FANCF gene listed in Table 6, mutations of the FANCG gene listed in Table 7.

The present invention also pertains to methods of determining if a patient has cancer, or is at increased risk of developing cancer, including pancreatic cancer, comprising the steps of: (a) providing a DNA sample from said patient; (b) amplifying the FANC gene from said patient with the FANC gene-specific polynucleotide primers; (c) sequencing the amplified FANC gene; and (d) comparing the FANC gene sequence from said patient to a reference FANC gene sequence, where a discrepancy between the two gene sequences indicates the presence of a cancer-associated coding change; wherein the presence of one or more cancer-associated coding changes indicates said patient has cancer or is at an increased risk of developing cancer.

In a preferred embodiment, the cancer-associated coding change in the FANCC gene is selected from the group consisting of: mutations of the FANCA gene listed in Table 2, mutations of the FANCC gene listed in Table 3, mutations of the FANCD2 gene listed in Table 4, mutations of the FANCE gene listed in Table 5, mutations of the FANCF gene listed in Table 6, mutations of the FANCG gene listed in Table 7.

The invention also relates to methods of treating a patient having cancer or having a risk of developing cancer who has one or more cancer-associated coding changes in the FANC genes comprising the step of administering a therapeutically effective amount of a chemotherapeutic DNA cross-linking agent. Also, the therapeutically effective amount of a chemotherapeutic DNA cross-linking agent can be a low dose compared to standard dosages (i.e., daily doses at one-twentieth to one-fifteenth the standard dose).

In a preferred embodiment of the method for treating a cancer patient or patient at risk of developing cancer, the cancer-associated coding change in the FANC gene is selected from the group consisting of: mutations of the FANCA gene listed in Table 2, mutations of the FANCC gene listed in Table 3, mutations of the FANCD2 gene listed in Table 4, mutations of the FANCE gene listed in Table 5, mutations of the FANCF gene listed in Table 6, mutations of the FANCG gene listed in Table 7.

The present invention also pertains to methods of screening for a cancer therapeutic, the method comprising the steps of: (a) providing one or more cells containing one or more cancer associated coding changes in the FANC genes; (b) growing said cells in the presence of a potential cancer therapeutic; and (c) determining the rate of growth of said cells in the presence of said potential cancer therapeutic relative to the rate of growth of equivalent cells grown in the absence of said potential cancer therapeutic; wherein a reduced rate of growth of said cells in the presence of said potential cancer therapeutic, relative to the rate of growth of equivalent cells grown in the absence of said potential cancer therapeutic, indicates that the potential cancer then is a cancer therapeutic.

The present invention also relates to a kit for detecting cancer-associated coding changes in a FANC gene comprising a polynucleotide primer pair specific for the FANC gene; a reference FANC gene sequence and packaging materials.

Thus, the present invention provides diagnostic and therapeutic methods for the treatmant of cancer, including pancreatic cancer.

### BRIEF DESCRIPTION OF THE DRAWINGS

FIG. 1: Screen for Fanconi Anemia defects by Fancd2 monoubiquitination assay. Equal cell numbers were untreated, or incubated with MMC for 18-20 hours, or irradiated with 15 Gy and incubated for 2 hours, after which protein lysates were made. Protein lysates were immunoblotted for Fancd2. Lack of the upper band indicates a defect in the proximal Fanconi pathway.

FIG. 2: Homozygous deletion of exons 7-14 in pancreatic cancer cell line PL11. DNA from pancreatic cancer cell line B×PC3 was used as a control; exons for both samples were amplified in the same PCR plate. Independent reactions were used to confirm the deletion in PL11 and in the parallel xenograft PX192.

FIG. 3: FA-defective cell lines are hypersensitive to crosslinking agents. a. MMC sensitivity of pancreatic cancer cell lines as measured by population quantitation using a measurement of total DNA. b. Cisplatin sensitivity of pancreatic cancer cell lines by DNA quantitation. c. MMC sensitivity of pancreatic cancer cell lines as measured by manual cell counts. d. MMC sensitiviy of HNSCC cell lines by DNA quantitation. e. Cisplatin sensitivity of HNSCC cell lines by DNA quantitation. Legends are consistent throughout a.-c. and d.-e.

FIG. 4: FA-defective cancer cell lines arrest in G2/M 48 hours after low concentrations of MMC. Cells were treated with various concentrations of MMC for 2 hours, and incubated without MMC for 48 hours, after which the cell cycle was analyzed using a flow cytometer.

FIG. 5 represents the reference amino acid sequence (a) and the reference nucleotide sequence (b) for FANCA.

FIG. 6 represents the reference amino acid sequence (a) and the reference nucleotide sequence (b)for FANCC.

FIG. 7 represents the reference amino acid sequence (a) and the reference nucleotide sequence (b) for FANCD2.

FIG. 8 represents the reference amino acid sequence (a) and the reference nucleotide sequence (b) for FANCE.

FIG. 9 represents the reference amino acid sequence (a) and the reference nucleotide sequence (b) for FANCF.

FIG. 10 represents the reference amino acid sequence (a) and the reference nucleotide sequence (b) for FANCG.

### DETAILED DESCRIPTION OF THE INVENTION

The FANC proteins A, C, E, F and G assemble in a multisubunit nuclear complex which results in the monoubiquitination of FANCD2 and play a role in homologous recombination repair of double stranded breaks. Double strand breaks produced during the repair of mitomycin interstrand crosslinks accumulate in the absence of an intact homologous repair system in cells deficient in members of the FANC complex.

The present invention is based on the discovery that the FANC gene mutations that exist in pancreatic cancers can define a therapeutically distinct patient group, in essence serving as an Achilles heel of the tumor. Cells from Fanconi anemia patients, BRCA2-null cells and cells defective in homology-directed DNA strand-break repair are reproducibly and exquisitely more sensitive to mitomycin C, other DNA interstrand cross-linking agents (such as cisplatin), and other inducers of DNA damage than are cells containing an intact FANC/BRCA2/Rad 51 repair system. This hypersensitivity to cross-linking drugs can provide an expanded therapeutic window, allowing the use of low-dose, low toxicity, and long-term rational chemotherapeutic options for the treatment of the FANC/BRCA2-related subset of pancreatic cancer patients. Occasional complete remissions of pancreatic cancer have been reported with therapies that included mitomycin. FANC gene status can be used as a standard laboratory determination in the care of pancreatic cancer patients. The use of prophylactic chemotherapy to eliminate neoplastic cells having loss of the remaining wild-type gene, in carriers of FANC and BRCA2 mutations that have not yet been diagnosed with cancer can also represent a means of treating patients at risk for developing cancer.

Cells lacking FANC gene function are hypersensitive to mitomycin C and other interstrand DNA-crosslinking chemicals. Cancers in FANC gene heterozygotes have lost the wild-type allele, and are likely to be hypersensitive to the same chemicals. Tumors having genetic inactivation of FANC genes cannot regain those genes; they are gone forever, thus preventing the emergence of resistant tumor cells. Low-dose mitomycin C (or other interstrand-crosslinking drugs) can be used to treat FANC-mutant tumors to take advantage of this hypersensitivity. At these doses (for example, daily doses at one-twentieth to one-fiftieth the standard dose), normal cells would experience no toxicity, but the cancer cells would.

There is evidence that cells are most sensitive (damaged) by interstrand DNA crosslinks when they are in S phase, synthesizing DNA. Most cancer cells, on any particular day, however, are not in S phase. This may be why conventional therapy often fails (even in FANC gene heterozygotes). The use of low-dose drug therapy would allow the continuous (or closely-spaced) drug dosing over a period of weeks or months, in contrast to the conventional use of mitomycin C for brief or single exposures. Thus, all cells would become exposed in S phase, and exhibit the hypersensitivity to the drug. Thus, FANC-heterozygous cancer patients could be treated with nontoxic and more effective therapy directed specifically to the chemical hypersensitivity of their tumor.

A panel of pancreatic cancer xenografts and cell lines have been analyzed for mutations in FANCC and FANCG. Several variants have been identified, including a homozygous germline nonsense mutation in FANCG (E105ter), a homozygous somatic frameshift deletion in FANCC and several amino acid changes (Table 1).

TABLE 1
Table 1: Variant Fanconi genes in pancreatic cancer.
^{S}Somatic, ^{G}Germline, as determined by sequencing of normal
DNA, or by previous reports in FA patients or normals.
M350V and E521K were not reported previously.

| | | |
|---|---|---|
| Cancer | FANCC/FANCG Coding Change | BRCA2 Coding Change |

FANCC

| | |
|---|---|
| PX102 | Frameshift exon 14 (LOH)^{S} |
| PX19 | D195V (LOH)^{G} |
| Su86.86 | M350V (LOH) |
| CAPAN2 | E521K (heterozygous) |

FANCG

| | | |
|---|---|---|
| Hs766T | E105ter (LOH)^{G} | |
| CAPAN1 | S7F (LOH)^{G} | 6174delT (LOH)^{G} |

The FA-defective cell lines CAPAN1, PL11 and Hs766T are all hypersensitive to MMC, as compared to other pancreatic and HNSCC cancer cell lines. These findings provide an explanation for the discovery that a subset of pancreatic cancers is highly sensitive to MMC-containing regimens. Now, pancreatic cancers can be genetically tested for defects in the pathways that repair interstrand crosslinks, such as the FA pathway. Patients with a defect in one of the repair pathways could then be treated rationally with crosslinking agents, possibly at a much lower dose than is customary.

While pancreatic cancer remains the only form of cancer (in non-FA patients) known to harbor ‘upstream’ FA pathway mutations to date, mutations in this pathway are unlikely to be restricted to cancers of the pancreas. Two ovarian cancer cell lines were recently shown to be defective in the FA pathway, which was attributed to FANCF-methylation (Taniguchi, T. et al. Disruption of the Fanconi anemia-BRCA pathway in cisplatin-sensitive ovarian tumors. Nat Med 9, 568-74. (2003).

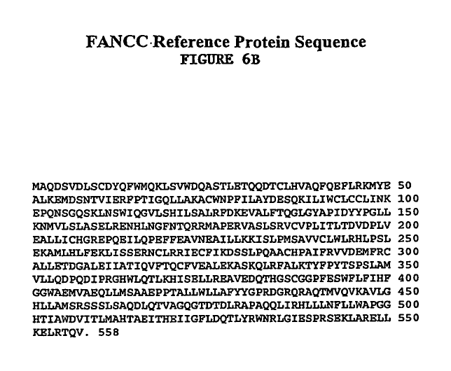

In addition to the coding changes and mutations mentioned in Table 1, there are many other coding changes and/or mutations in FANC genes that have been identified (see Tables 2, 3, 4, 5, 6 and 7 herein). These coding changes and mutations are listed in the Fanconi Anemia Mutation Database that is maintained and continuously updated by The Rockefeller University (see www.rockerfeller.edu/fanconi/mutate/). The listed coding changes/mutations are identified when compared to the FANC gene and proteins reference sequences found in FIGS. 5 thru 10. Any one or more of these coding changes or mutations can be used for determining if a patient has cancer or is at increased risk of developing cancer, particularly pancreatic cancer. Once identified, the patient with a FANC gene coding change/mutation can be treated prophalactically or therapeutically for cancer.

The coding changes and mutations listed in the Fanconi Anemia Mutation Database as of the filing date of this application are listed below. It is intended that the methods of the present invention will include FANC genes with coding changes and/or mutations that have been identified but not listed in the Fanconi Anemia Mutation Database or will be listed in the future.

TABLE 2
FANCA Coding Changes/Mutations

| Mutation | Type |
|---|---|
| 1-4368del | deletion |
| exon 24-28del | unknown |
| 24C>G | AA substitution |
| 65G>A | stop codon |
| 1-596del | |
| 1A>G | AA substitution |
| 154C>T | stop codon |
| 401insC | frameshift |
| 542C>T | AA substitution |
| IVS6-2A>G | deletion |
| IVS7+1G>A | RNA splicing |
| 597-3066del | deletion |
| 732G>C | AA substitution |
| 755A>G | AA substitution |
| 790C>T | stop codon |
| 795-808del | frameshift |
| IVS10+1G>T | RNA splicing |
| 827-1225del | deletion |
| 856C>T | stop codon |
| 890-893del | frameshift |
| RNA splicing | RNA splicing |
| 987-990del | frameshift |
| 894-1359del | frameshift |
| 1164-1165del | frameshift |
| 1303C>T | AA substitution |
| IVS14+1G>C | RNA splicing |
| 1459insC | frameshift |
| 1475A>G | AA substitution |
| 1771C>T | stop codon |
| 2005C>T | stop codon |
| 1944delG | frameshift |
| 2066delG | frameshift |
| 2314C>T | stop codon |
| 2450T>C | AA substitution |
| IVS26 +134A>G | frameshift |
| 2535-2536del | frameshift |
| 2534T>C | AA substitution |
| 2524delT | frameshift |
| 2546delC | frameshift |
| IVS27 -1G>A | deletion |
| IVS27 -2A>T | deletion |
| IVS27 -2A>T | RNA splicing |
| IVS29 -19 del19 | RNA splicing |
| 2840C>G | stop codon |
| 2982-3066del | frameshift |
| 3164G>T | AA substitution |
| 3188G>A | stop codon |
| 3091C>T | stop codon |
| 3163C>T | AA substitution |
| 3382C>G | AA substitution |
| 3349A>G | AA substitution |
| 3391A>G | AA substitution |
| 3396-3399del | frameshift |
| 3520-3522del | deletion |
| 3559insG | frameshift |
| 3760G>T | AA substitution |
| 3788-3790del | deletion |
| 3904T>C | AA substitution |
| 3982A>G | AA substitution |
| IVS40+1-18del | exon skip |
| 4069-4082del | frameshift |
| IVS41 -2A>G | deletion |
| 4275delT | frameshift |
| 1-2981del | |
| 1007-3066del | deletion |
| 1115-1118del | Frameshift |
| 1191-1194del | frameshift |
| 1360-1626del | deletion |
| 1360-1826del | frameshift |
| 1471-1826del | deletion |
| 1471-1626del | Deletion |
| 1615delG | frameshift |
| 163C>T | stop codon |
| 1627-1900del | frameshift |
| 1827-2778del | deletion |
| 1901-2778del | Deletion |
| 2107C>T | stop codon |
| 2167-2169del | deletion |
| 2495-2497del | deletion |
| 2574C>G | AA substitution |
| 2779-3066del | deletion |
| 2779-3348del | deletion |
| 2830ins19 | frameshift |
| *2982-4365del | deletion |
| 3061-3154del | frameshift |
| 3263C>T | AA substitution |
| 3329A>C | AA substitution |
| 3398delA | frameshift |
| 3403-3405del | deletion |
| 3629-3630insT | frameshift |
| 3715-3729del | Deletion |
| 3760-3761del | Frameshift |
| 3786C>G | AA substitution |
| 3884T>A | stop codon |
| 3920delA | frameshift |
| 3971C>T | AA substitution |
| 4010delG+18 | exon skip |
| 4015delC | frameshift |
| 4080G>C | AA substitution |
| 4267-4404del | unknown |
| *427-522*del | deletion |
| 4249C>G | AA substitution |
| *5′UTR-1900*del | deletion |
| *5′UTR-3066del | deletion |
| *5′UTR-522*del | deletion |
| 523-1359del | deletion |
| 597-1826del | deletion |
| IVS7+5G>A | RNA splicing |
| IVS7+5G>T | RNA splicing |
| 862G>T | AA substitution |
| 894-1006del | frameshift |
| ex10-12del* | deletion |
| ex10-17del | deletion |
| 4075G>T | AA substitution |
| IVS10-1G>A | RNA splicing |
| IVS15-1G>T | RNA splicing |
| IVS16+3A>C | exon skip |
| IVS38-1G>C | RNA splicing |
| IVS9+3del | frameshift |

TABLE 3
FANCC CODING CHANGES/MUTATIONS

| Mutation | Type |
|---|---|
| 1806insA | Insertion |
| 322delG | Frameshift |
| 775C>T | AA substitution |
| IVS4+4A>T | RNA splicing |
| 1742T>G | AA substitution |
| 1916T>C | AA substitution |
| 292C>T | Stop codon |
| 808C>T | stop codon |
| 1897C>T | Stop codon |
| 320G>A | Stop codon |

TABLE 4
FANCD2 Coding Changes/Mutations

| Mutation | Type |
|---|---|
| 3707G>A | AA substitution |
| 376A>G | RNA splicing |
| 904C>T | AA substitution |
| 958C>T | stop codon |
| Exon 17del | exon skip |

TABLE 5
FANCE Coding Changes/Mutations

| Mutation | Type |
|---|---|
| 3707G>A | AA substitution |
| 376A>G | RNA splicing |
| 904C>T | AA substitution |
| 958C>T | stop codon |
| Exon 17del | exon skip |

TABLE 6
FANCF Coding Changes/Mutations

| Mutation | Type |
|---|---|
| 16C>T | stop codon |
| 230-252del | deletion |
| 327C>G | stop codon |
| 349-395del | deletion |
| 484-485del | frameshift |

TABLE 7
FANCG CODING CHANGES/MUTATIONS

| Mutation | Type |
|---|---|
| IVS3+1G>C | RNA splicing |
| 244-245insG | frameshift |
| 313G>T | stop codon |
| 1066C>T | stop codon |
| IVS8-2A>G | RNA splicing |
| IVS11+1G>C | RNA splicing |
| 1649delC | frameshift |
| 1642C>T | stop codon |
| 109-110del | frameshift |
| 1183-1192del | frameshift |
| 1310-1311insGA | frameshift |
| 1636G>C | AA substitution |
| 1715G>A | stop codon |
| 1749delA | frameshift |
| 212T>C | AA substitution |
| 346-347del | frameshift |
| 652C>T | stop codon |
| IVS13-1G>C | RNA splicing |
| IVS2+1G>A | exon skip |
| IVS5+1G>T | RNA splicing |
| IVS9-1G/C | RNA splicing |

Definitions:

“Chemotherapeutic DNA Cross-Linking Agents” refer to a class of drugs useful for chemotherapy are nucleophillic materials capable of being absorbed into the nucleus of a cell endocytotically and there cross-linking the DNA, referred to hereinafter as DNA cross-linking agents. These cross-linking agents are bifunctional compounds, possessing at least two nucleophillic centers capable of covalently bonding to electrophyllic centers in the DNA macromolecule. Cross-linking the DNA in the cell serves to kill the cell as it no longer has normal nuclear functions. The DNA reactive cross-linking chemotherapeutic agents referred to in this application are agents well known in the art. These agents are bifunctional electrophillic or reactive compounds capable of tightly binding to the DNA macromolecule. Included in this class, besides the nitrogen mustard, are other nitrogen mustards such as galactose mustard, L-phenylalanine mustard and cyclophosphoamide mustard, as well as compounds such as formamide, doxorubicin, amphotericin B, mitomycin, 1,3-bis(2-chloroethyl)-1-nitrosourea (carmustine), thio TEPA, dimethyl myleran, trimethyldamine, and numerous others.

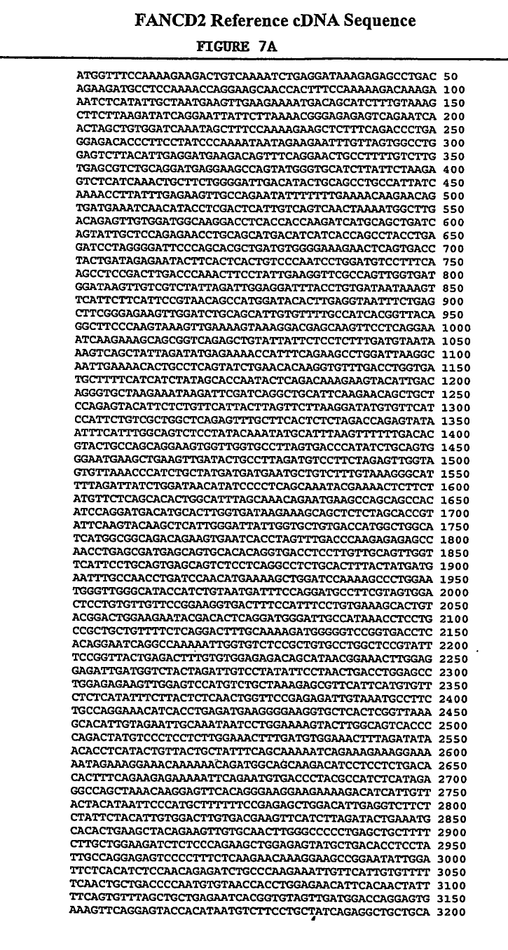

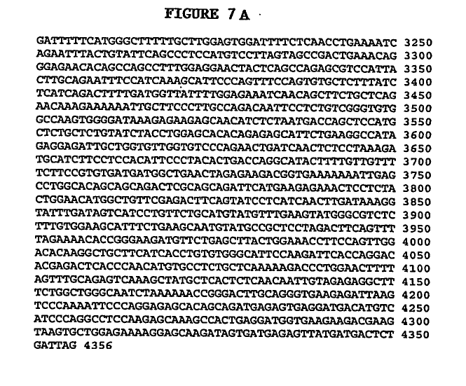

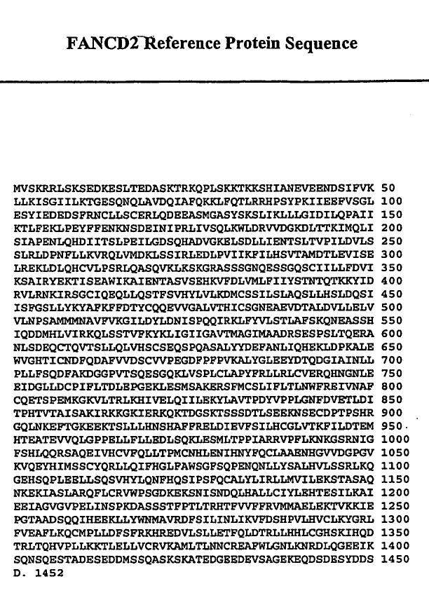

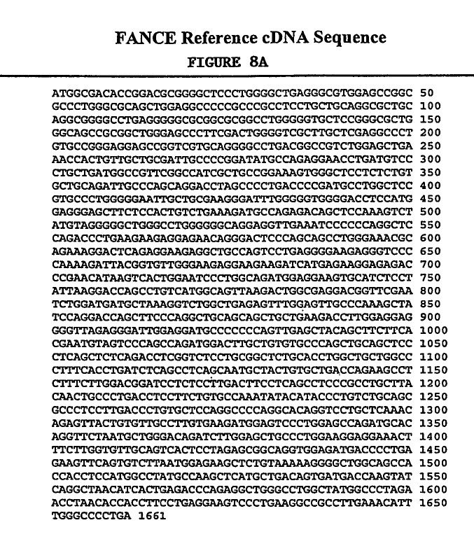

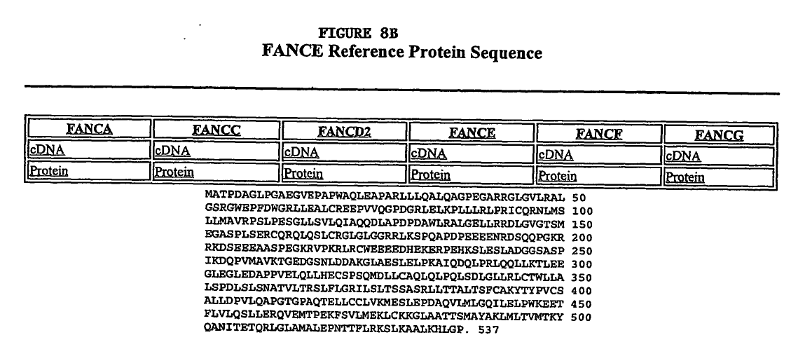

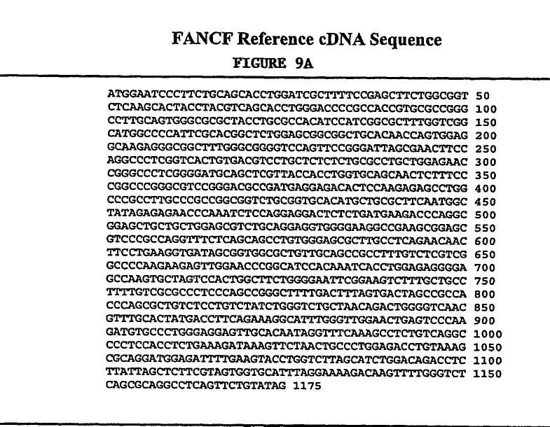

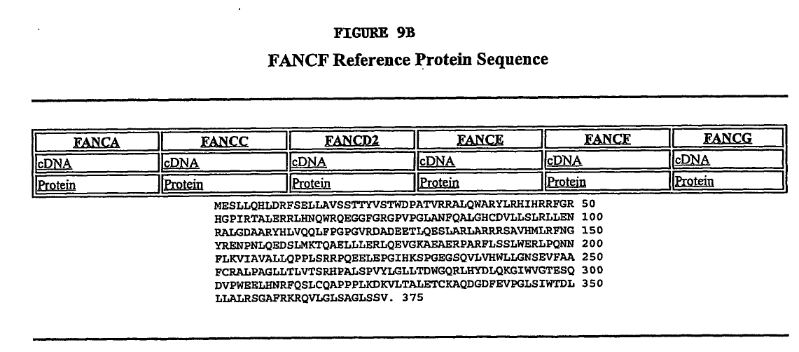

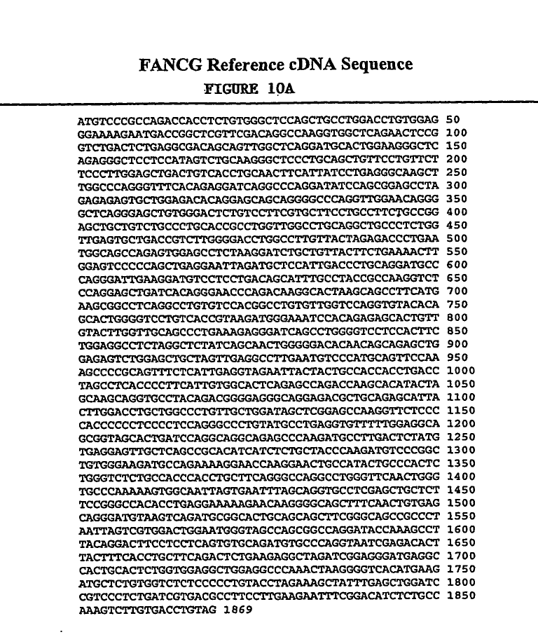

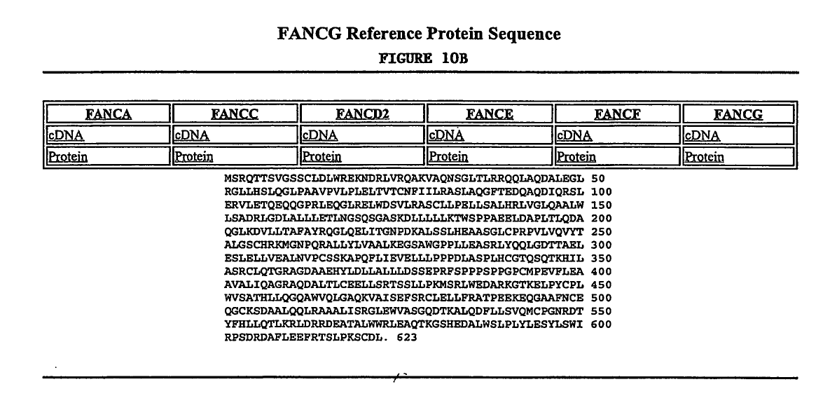

“FANC Genes” refer to nucleic acids and polypeptides having sequences or homologous sequences to the following: 1) FANC-A (e.g., Genbank Accession No.: NM_{—}000135; also see FIG. 5) 2) FANC-B (not yet cloned) 3) FANC-C (e.g., Genbank Accession No.: NM_{—}000136; also see FIG. 6) 4) FANC-D1/ (e.g., Genbank Accession No.: U43746) BRCA-2 5) FANC-D2 (e.g., Genbank Accession No.: NM_{—}033084; also see FIG. 7) 6) FANC-E (e.g., Genbank Accession No.: NM_{—}021922) 7; also see FIG. 8) FANC-F (e.g., Genbank Accession No.: NM_{—}022725; also see FIG. 9) 8) FANC-G (e.g., Genbank Accession No.: BC000032; also see FIG. 10) 9) BRCA-1 (e.g., Genbank Accession No.: U14680) 10) ATM (e.g., Genbank Accession No.: U33841)”.

“At Risk” or “Increased Risk” refers to the greater incidence of cancer in those patients having altered FANC genes or proteins as compared to those patients without alterations in the FANC pathway genes or proteins. “Increased risk” also refers to patients who are already diagnosed with cancer and may have an increased incidence of a different cancer form. According to the invention, “increased risk” of cancer refers to cancer-associated coding change in a FANC/BRCA pathway gene that contributes to a 50%, preferably 90%, more preferably 99% or more increase in the probability of acquiring cancer relative to patients who do not have a cancer-associated coding change in a FANC/BRCA pathway gene.

“Coding Change” refers to a change in nucleotide sequence within a gene, or outside the gene in a regulatory sequence compared to wild type. The change may be a deletion, substitution, point mutation, mutation of multiple nucleotides, transposition, truncation, termination, inversion, frame shift, nonsense mutation or other forms of aberration that differentiate the nucleic acid or protein sequence from that of a normally expressed gene in a functional cell where expression and functionality are within the normally occurring range.

“Amplifying” when applied to a nucleic acid sequence refers to a process whereby one or more copies of a particular nucleic acid sequence is generated from a template nucleic acid, preferably by the method of polymerase chain reaction (Mullis and Faloona, 1987, Methods Enzymol., 155:335). “Polymerase chain reaction” or “PCR” refers to an in vitro method for amplifying a specific nucleic acid template sequence. The PCR reaction involves a repetitive series of temperature cycles and is typically performed in a volume of 50-100 .mu.1. The reaction mix comprises dNTPs (each of the four deoxynucleotides dATP, dCTP, dGTP, and dTTP), primers, buffers, DNA polymerase, and nucleic acid template. The PCR reaction comprises providing a set of polynucleotide primers wherein a first primer contains a sequence complementary to a region in one strand of the nucleic acid template sequence and primes the synthesis of a complementary DNA strand, and a second primer contains a sequence complementary to a region in a second strand of the target nucleic acid sequence and primes the synthesis of a complementary DNA strand, and amplifying the nucleic acid template sequence employing a nucleic acid polymerase as a template-dependent polymerizing agent under conditions which are permissive for PCR cycling steps of (i) annealing of primers required for amplification to a target nucleic acid sequence contained within the template sequence, (ii) extending the primers wherein the nucleic acid polymerase synthesizes a primer extension product. “A set of polynucleotide primers” or “a set of PCR primers” can comprise two, three, four or more primers.

Other methods of amplification include, but are not limited to, ligase chain reaction (LCR), polynucleotide-specific base amplification (NSBA), or any other method known in the art.

“Polynucleotide Primer” refers to a DNA or RNA molecule capable of hybridizing to a nucleic acid template and acting as a substrate for enzymatic synthesis under conditions in which synthesis of a primer extension product which is complementary to a nucleic acid template is catalyzed to produce a primer extension product which is complementary to the target nucleic acid template. The conditions for initiation and extension include the presence of four different deoxyribonucleoside triphosphates and a polymerization-inducing agent such as DNA polymerase or reverse transcriptase, in a suitable buffer (“buffer” includes substituents which are cofactors, or which affect pH, ionic strength, etc.) and at a suitable temperature. The primer is preferably single-stranded for maximum efficiency in amplification. “Primers” useful in the present invention are generally between about 10 and 35 nucleotides in length, preferably between about 15 and 30 nucleotides in length, and most preferably between about 18 and 25 nucleotides in length.

“Cancer-Associated Coding Change” refers to any sequence change in the amino acid sequence of a protein encoded by a FANC/BRCA gene, as defined herein, harbors a defect, as defined herein, that can cause or is associated with a cancer in a patient.

“Defect” refers to any alteration of a gene or protein within the FANC/BRCA pathway, and/or proteins, with respect to any unaltered gene or protein within the FANC/BRCA pathway.

“Tumor” refers to a neoplasm that may either be malignant or non-malignant. Tumors of the same tissue type originate in the same tissue, and may be divided into different subtypes based on their biological characteristics.

“Cancer” refers to a malignant disease caused or characterized by the proliferation of cells which have lost susceptibility to normal growth control. “Malignant disease” refers to a disease caused by cells that have gained the ability to invade either the tissue of origin or to travel to sites removed from the tissue of origin.

A patient is “treated” according to the invention if one or preferably more symptoms of cancer as described herein are eliminated or reduced in severity, or prevented from progressing or developing further.

“Therapeutically effective amount” refers to the total amount of each active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions.

“A reduced growth rate” refers to a decrease of 50%, preferably 90%, more preferably 99% and most preferably 100% in the rate of cellular proliferation of a tumor cell line with a FANC/BRCA cancer-associated coding change that is being treated with a potential therapeutic agent relative to cells of a the same tumor cell line that is not being treated with a potential therapeutic agent.

“Microarray”, or “Array”, refers to a plurality of unique biomolecules attached to one surface of a solid support. Preferably, a biomolecule of the invention a potential therapeutic agent as described herein. In this embodiment, the microarray of the invention comprises nucleic acids, proteins, polypeptides, peptides, fusion proteins or small molecules that are immobilised on a solid support, generally at high density. Each of the biomolecules is attached to the surface of the solid support in a pre-selected region. Suitable solid supports are available commercially, and will be apparent to the skilled person. The supports may be manufactured from materials such as glass, ceramics, silica and silicon. The supports usually comprise a flat (planar) surface, or at least an array in which the molecules to be interrogated are in the same plane. In one embodiment, the array is on microbeads. In one embodiment, the array comprises at least 10, 500, 1000, 10,000 different biomolecules attached to one surface of the solid support.

In the present application, nucleotide names are listed in the tables of the application in abbreviated form, for example, “A” for adenine; “G” for guanine, “T” for thymine, ‘C” for cytosine and “U” for uracil. The terminology used for identifying nucleotide positions/ substitutions/ deletions is illustrated as follows: 862G>T indicates that the guanine base at position 862 has been replaced with a thymine. In addition, other abbreviations used in the tables of the application include “ex” representing exon(s), “del” representing deletion and “ins” representing insertion. Other terminology used in the tables of the application are well-known to those skilled in the art. In terms of determining the coding changes/mutations of FANC genes listed in the Fanconi Anemia Mutation Database cited above reference is made to the reference nucleotide and amino acid sequences found in FIGS. 5 thru 10 of the present application.

Pharmaceutical Compositions

Another aspect of the invention pertains to pharmaceutical compositions of the chemotherapeutic DNA cross-linking agents useful in the methods of the invention. The pharmaceutical compositions of the invention typically comprise a compound useful in the methods of the invention and a pharmaceutically acceptable carrier. As used herein “pharmaceutically acceptable carrier” includes any and all solvents, dispersion media, coatings, antibacterial and antifungal agents, isotonic and absorption delaying agents, and the like that are physiologically compatible. The type of carrier can be selected based upon the intended route of administration. In various embodiments, the carrier is suitable for intravenous, intraperitoneal, subcutaneous, intramuscular, topical, transdermal or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, the compounds can be administered in a time release formulation, for example in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are generally known to those skilled in the art.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Depending on the route of administration, the compound may be coated in a material to protect it from the action of enzymes, acids and other natural conditions which may inactivate the agent. For example, the compound can be administered to a subject in an appropriate carrier or diluent co-administered with enzyme inhibitors or in an appropriate carrier such as liposomes. Pharmaceutically acceptable diluents include saline and aqueous buffer solutions. Enzyme inhibitors include pancreatic trypsin inhibitor, diisopropylfluoro-phosphate (DEP) and trasylol. Liposomes include water-in-oil-in-water emulsions as well as conventional liposomes (Strejan, et al., (1984) J. Neuroimmunol 7:27). Dispersions can also be prepared in glycerol, liquid polyethylene glycols, and mixtures thereof and in oils. Under ordinary conditions of storage and use, these preparations may contain a preservative to prevent the growth of microorganisms.

The active agent in the composition (i.e., chemotherapeutic DNA cross-linking agent) preferably is formulated in the composition in a therapeutically effective amount. A “therapeutically effective amount” refers to an amount effective, at dosages and for periods of time necessary, to achieve the desired therapeutic result to thereby influence the therapeutic course of a particular disease state. A therapeutically effective amount of an active agent may vary according to factors such as the disease state, age, sex, and weight of the individual, and the ability of the agent to elicit a desired response in the individual. Dosage regimens may be adjusted to provide the optimum therapeutic response. A therapeutically effective amount is also one in which any toxic or detrimental effects of the agent are outweighed by the therapeutically beneficial effects. In another embodiment, the active agent is formulated in the composition in a prophylactically effective amount. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The amount of active compound in the composition may vary according to factors such as the disease state, age, sex, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

A compound of the invention can be formulated into a pharmaceutical composition wherein the compound is the only active agent therein. Alternatively, the pharmaceutical composition can contain additional active agents. For example, two or more compounds of the invention may be used in combination.

This invention is further illustrated by the following examples which should not be construed as limiting. The contents of all references, patents, and published patent applications cited throughout this application, as well as the figures, are incorporated herein by reference.

Exemplification

Methods and Materials:

The following materials and methods were used in Examples 1-3 unless otherwise noted.

Samples

For the FA pathway screen by Fancd2 immunoblot, we included breast and prostate cancer cell lines because of the role of BRCA2 in familial breast cancer and young-onset prostate cancer (Edwards, S. M. et al. Two percent of men with early-onset prostate cancer harbor germline mutations in the BRCA2 gene. Am J Hum Genet 72, 1-12, 2003; Cancer risks in BRCA2 mutation carriers. The Breast Cancer Linkage Consortium. J Natl Cancer Inst 91, 1310-6, 1999). Because of the occurrence of HNSCC in a substantial percentage of FA patients who survive until adulthood (Kutler, D. I. et al. High incidence of head and neck squamous cell carcinoma in patients with Fanconi anemia. Arch Otolaryngol Head Neck Surg 129, 106-12, 2003), we included eight HNSCC cell lines. A number of cell lines were previously tested by the National Cancer Institute for sensitivity to various chemotherapeutic agents (http://dtp.nci.nih.gov); the two cell lines most sensitive to MMC (SW-1088, astrocytoma and NCI-H460, large cell lung cancer) were obtained from ATCC (American Type Culture Collection, Manassas, Va.) and included in our panel. Pancreatic cancer cell lines MiaPaCa2, BxPC3, Panc-1, AsPC1, Su86.86, CFPAC, CAPAN1, CAPAN2, Hs766T, Hpaf II, Colo357, Mpanc96; breast cancer cell lines MDAMB 175-VII, MDAMB 231, MDAMB 361, MDAMB 436, MDAMB 453, MDAMB 461, MDAMB 468, BT 474, BT 549, ZR75-1, ZR75-30, SKBR3, MCF7, HS578; head and neck cancer cell lines Detroit 562, FaDu, SCC-15, SCC-25, Cal27, RPMI-2650, A-253, SW-579 and prostate cancer cell lines MDA Pca-2b, DU145, PC3, LNcap were obtained from the ATCC and ECACC (European Collection of Animal Cell Cultures, Salisbury, UK). Prostate cancer cell lines C2-4B and CWR22 were kindly provided by Dr A. M. DeMarzo (Department of Pathology, Johns Hopkins University). Pancreatic cancer cell lines Panc 3.27 (PL11), Panc 6.03, Panc 8.13, Panc 2.03, Panc 2.13, Panc 1.28, Panc 4.21, Panc 5.04, PL3, PL5, PL6 and PL13 were kindly provided by Dr. E. M. Jaffee (Department of Oncology, Johns Hopkins University); PL45 was created in our lab (Caldas, C. et al. Frequent somatic mutations and homozygous deletions of the p16 (MTS1) gene in pancreatic adenocarcinoma. Nat Genet 8, 27-32, 1994). Panc 3.27, Panc 6.03, Panc 8.13, Panc 2.03, Panc 2.13 and PL45 are also available from ATTC. Cells were grown in media supplemented with 10% fetal bovine serum, penicillin/streptomycin and L-glutamine. Xenograft PX191 was established as previously described (Hahn, S. A. et al. Allelotype of pancreatic adenocarcinoma using xenograft enrichment Cancer Res 55, 4670-5, 1995).

Fancd2 Immunoblots

Equal numbers of cells were grown in 6-well plates and treated with or without MMC, 45 nM, for 18-24 hours, or irradiated with 15 Gy and incubated for 2 hours. Cells were lysed, boiled and loaded on 3-8% tris-acetate polyacrylamide gels Invitrogen, Carlsbad, Calif.). Protein was transferred onto a PVDF membrane and blocked for one hour in TBST (tris-buffered saline; Tween-20) 5% milk. Blots were incubated with mouse anti-Fancd2 antibody (sc20022, Santa Cruz Biotechnology, Santa Cruz, Calif.), diluted 1:1000, overnight at room temperature. Blots were washed with TBST and incubated with goat anti-mouse HRP. Binding was detected using Super Signal West Pico chemiluminescence substrate (Pierce Biotechnology, Rockford, Ill.).

Sequencing and Deletion Mapping

FANCC and FANCG were sequenced as described in: van der Heijden, M. S., Yeo, C. J., Hruban, R. H. & Kern, S. E. Fanconi anemia gene mutations in young-onset pancreatic cancer. Cancer Res 63, 2585-8. (2003).; FANCA, FANCE and FANCF were sequenced using automated sequencing. Primers for sequencing and for determination of the breakpoints of the homozygous deletion were purchased from IDT DNA (Coralville, Iowa).

Survival Studies

Picogreen: 1.2×10³ cancer cells per well were incubated with various concentrations of MMC (Sigma, Saint Louis, Mo.; range 0-4.5 μM) or cisplatin (Sigma; range 0-10 μM) in 96-well plates. Cells were incubated for a period of time long enough to allow non-treated cells to reach at least a threefold increase in fluorescence as compared to day 1 (3-7 days). Medium was changed every 48 hours. Cells were washed with PBS, and lysed in 100 μL sterile water. After 1 hour, 100 μl 0.5% Picogreen (Molecular Probes, Eugene, Oreg.) in tris-EDTA buffer was added to each well. After 45 minutes, wells were read in a fluorometer. Survival was calculated as a percentage; the wells without drugs were considered as 100 percent. Each experiment was done in duplicate; at least six experiments per cell line per concentration were performed.

Cell counts: 1×10⁵ cells were plated in tissue culture flasks (25 cm²). The next day, the medium was substituted with MMC-containing medium (range 0-4.5 μM). Cells were counted after 3-4 population doublings (4-7 days) using a hemacytometer. Four experiments per cell line/concentration were done.

Cell Cycle Analysis

Cells were cultured in 25 cm² flasks and treated with MMC for two hours. Cells were washed with PBS and incubated with normal tissue culture medium for 48 hours.

Cells were obtained by trypsinization and resuspended in 3.7% paraformaldehyde in PBS, stained with Hoechst 33258 (Sigma), incubated at 4° C. for 10 min and analyzed using a flow cytometer. A “G2/M arrest” was defined as a twofold increase of the percentage of cells in G2/M, as compared to untreated cells.

### EXAMPLE 1

### FA Pathway Defects Identified by Fancd2 Monoubiquitination

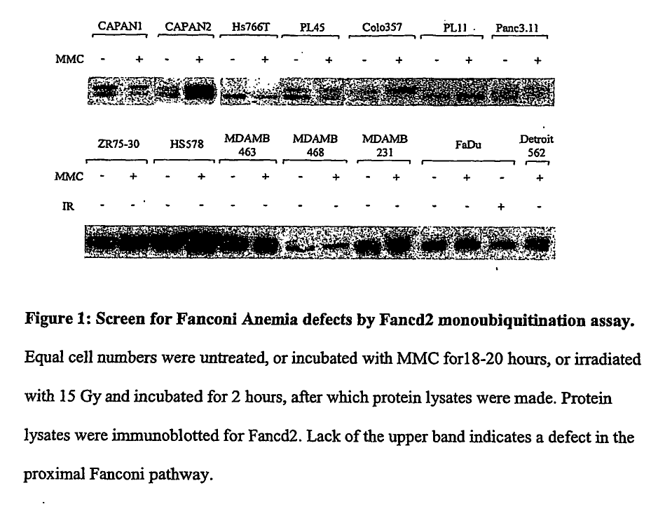

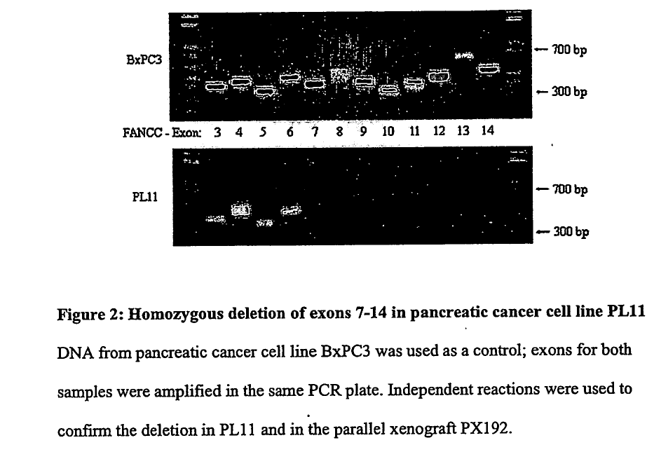

The FA proteins Fanca, Fancc, Fance, Fancf and Fancg assemble in a nuclear complex in response to DNA damage from crosslinking agents. This multiprotein complex is required for the monoubiquitination of Fancd2. Recently, evidence has been provided that PHF9 (FANDCL), another member of the Fanconi nuclear complex, has an important role in Fancd2 monoubiquitination (Meetei, A. R. et al. A novel ubiquitin ligase is deficient in Fanconi anemia. Nat Genet 35, 165-70, 2003). An immunoblot for Fancd2 after MMC treatment normally detects a short (Fancd2-S; 155 kD) and a long (Fancd2-L, mono-ubiquitinated; 162 kD) isoform. The presence of only the short band is indicative of a defect in the upstream FA pathway (Gregory, R. C., Taniguchi, T. & D'Andrea, A. D. Regulation of the Fanconi anemia pathway by monoubiquitination. Semin Cancer Biol 13, 77-82, 2003). To assess the pathogenicity of previously described changes in the FANCC and FANCG genes in pancreatic cancer cell lines (table 1), a Fancd2 immunoblot analysis of cells treated with MMC was used to analyze Hs766T, Su86.86, CAPAN1 and CAPAN2 cells (FIG. 1). Hs766T cells contained only the Fancd2-S isoform, indicating a defect in Fancd2 monoubiquitination. The other cell lines had normal Fancd2 monoubiquitination, indicating that the variants in these cell lines are not null alleles. The Brca2 protein functions downstream in the FA pathway or in a separate pathway with overlapping functions (D'Andrea, A. D. & Grompe, M. The Fanconi anaemia/BRCA pathway. Nat Rev Cancer 3, 23-34, 2003). CAPAN1 cells, carrying a mutation in BRCA2 (Goggins, M. et al. Germline BRCA2 gene mutations in patients with apparently sporadic pancreatic carcinomas. Cancer Res 56, 53604, 1996), were thus found to undergo Fancd2 monoubiquitination (FIG. 1).We next extended our functional test of the FA pathway to a panel of 21 pancreatic cancer cell lines, 14 breast cancer cell lines, 6 prostate cancer cell lines, 8 HNSCC cell lines, a glioma cell line and a lung cancer cell line (FIG. 1 and data not shown). Two additional cell lines were found to be defective in Fancd2 monoubiquitination: the pancreatic cancer cell line PL11 and the HNSCC cancer cell line FaDu. All defects were confirmed with separately prepared lysates. In the case of FaDu, a faint shadow above the short Fancd2 isoform was seen on each immunoblot, although an unambiguous monoubiquitinated band was never seen. Several additional Fancd2 immunoblots, including immunoblots on lysates after irradiation (FIG. 1) and on an aliquot separately purchased (data not shown) resulted in similar findings. A total of 8 FaDu-lysates (including untreated, MMC treated and irradiated samples) was examined; each of these samples failed to show a monoubiquitinated Fancd2-isoform. We next examined these cell lines for genetic defects in FANCC and FANCG, the only FA genes proximal to BRCA2 shown to date to be mutated in cancers in non-FA patients. The pancreatic cancer cell line PL11 had a deletion of eight exons of FANCC: exons 7-14 (FIG. 2). The deletion was further analyzed by PCR with additional primer sets: at the 5′end, the breakpoint was found to occur between IVS6+88 and IVS6+1018; at the 3′end the breakpoint was mapped down to a region between 15,057 and 20,846 basepairs downstream from the stopcodon. PL11 was derived independently from the same surgically resected cancer as was the xenograft PX192. Analysis of this xenograft showed the same homozygous deletion, proving that this homozygous deletion must have been present in the original tumor. A heterozygous polymorphism was encountered between exons 7 and 8 in normal DNA taken from the same patient, indicating that the deletion was somatic, with loss of the other allele. No mutations in FANCC and FANCG were found in FaDu. Next, we sequenced FANCA, FANCE and FANCF in FaDu: no mutations were found.

### EXAMPLE 2

### FA Defects are Associated with Increased Cytotoxicity by Crosslinking Agents

The FA-defective pancreatic cancer cell lines Hs766T (FANCG-mutated), PL11 (FANCC-mutated) and CAPAN1 (BRCA2-mutated) and FA-proficient cell lines Su86.86 and MiaPaCa2 were treated with various concentrations of either MMC or cisplatin, and incubated in 96-well plates. Relative cell numbers were determined by measurement of doublestranded DNA content using Picogreen; wells containing no compound were used as controls. The same experiments were done with the HNSCC cell lines FaDu (FA-defective), Detroit 562 and A-253. The FA-defective cell lines

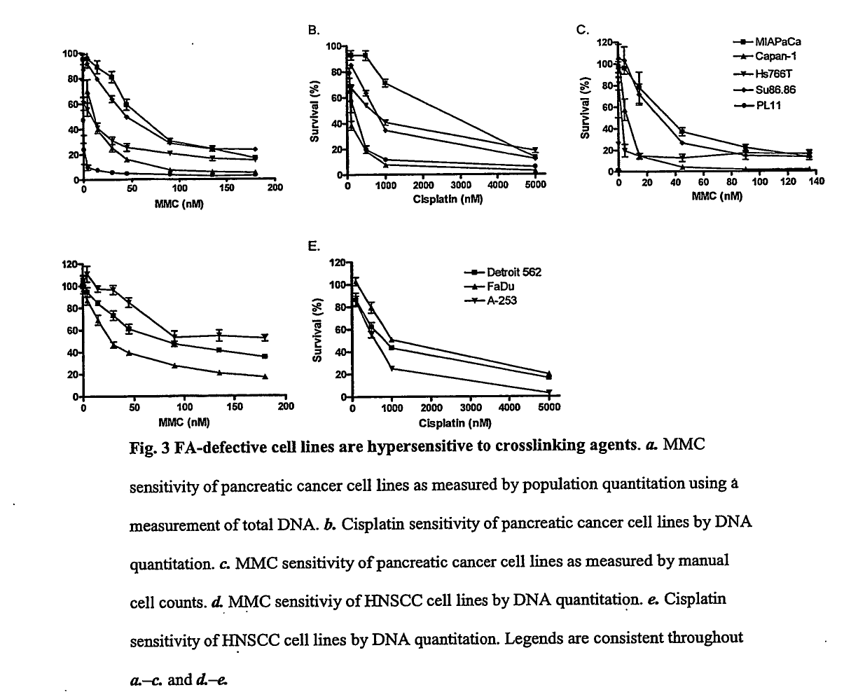

Hs766T, PL11 and CAPAN1 had an increased sensitivity to MMC, as compared to MiaPaCa2 and Su86.86 (FIG. 3A). CAPAN1 and PL11 are hypersensitive to cisplatin (FIG. 3B); Su86.86 and Hs766T were less sensitive than CAPAN1 and PL11, but had an increased sensitivity to cisplatin as compared to MiaPaCa2. To confirm the results obtained with the Picogreen assay, we also assessed sensitivity to MMC of the cell lines MiaPaCa2, Su86.86, CAPAN1 and Hs766T with manual (hemacytometer) cell counts. This assay confirmed their hypersensitivity to MMC (FIG. 3C). The FA-defective HNSCC cell line FaDu was also hypersensitive to MMC (FIG. 3D), but not to cisplatin (FIG. 3E), as compared to the FA-proficient HNSCC cell lines A-253 and Detroit 562.

### EXAMPLE 3

### G2/M Cell Cycle Arrest by Low-Dose Crosslinking Agents in FA-Defective Cancer Lines

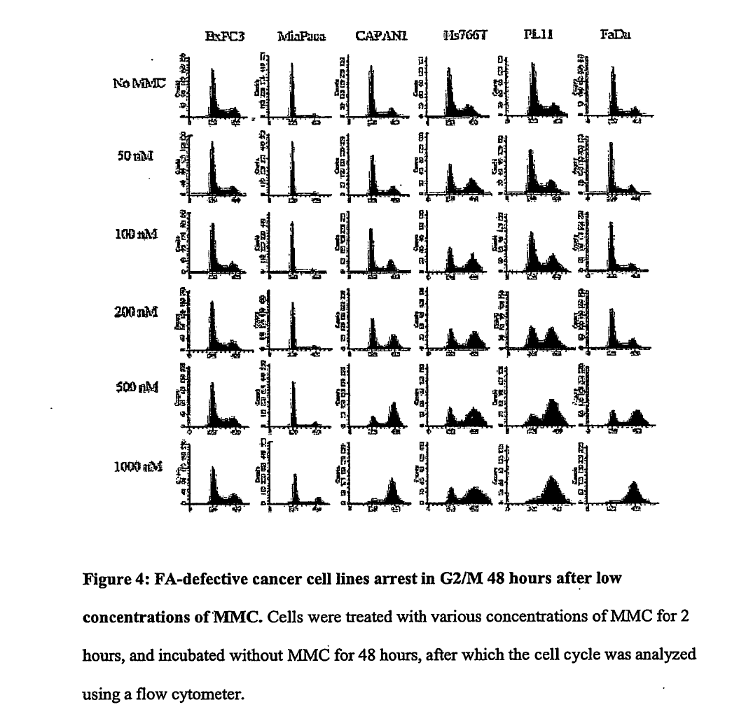

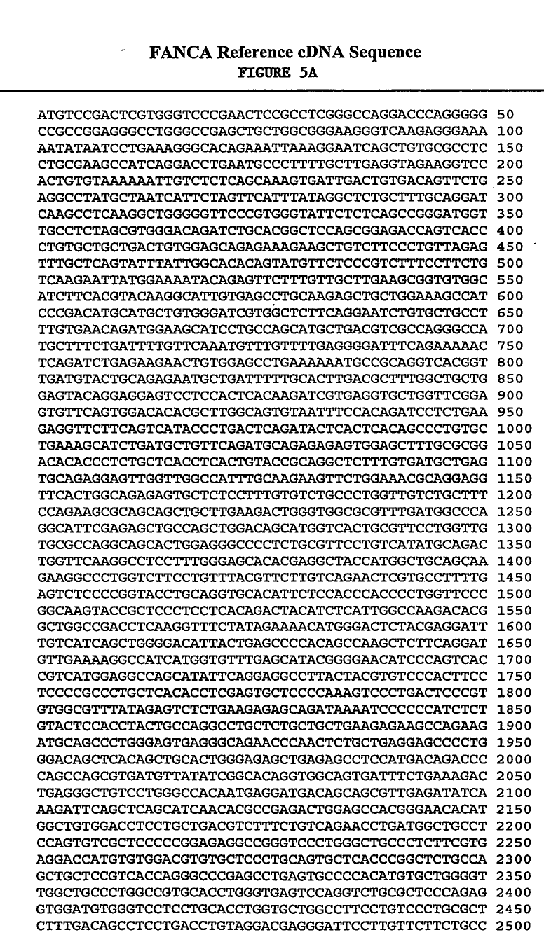

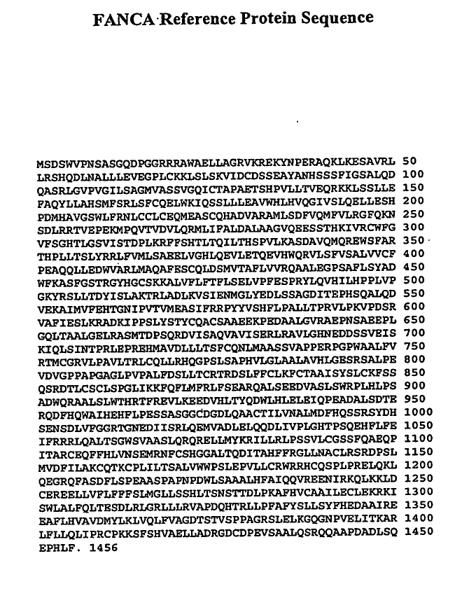

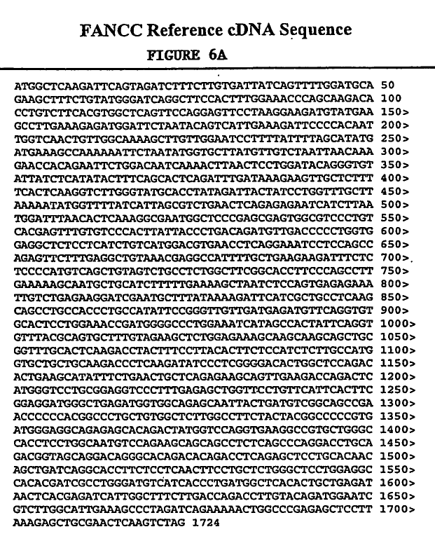

The methods used to investigate cell “survival” upon treatment with MMC and cisplatin sum the effects of cell death, slow growth and the occurrence of a cell cycle arrest. To determine the potential contribution of an arrest, we analyzed cell cycle distributions of DNA content after MMC treatment. Six pancreatic cancer cell lines (BxPC3, MiaPaCa2, Su86.86, PL11, Hs766T and CAPAN1) and one HNSCC cell line (FaDu) were analyzed 48 hours after MMC treatment for 2 hours; a G2/M arrest was defined as a twofold increase of the fraction of cells containing 4N DNA content, as compared to untreated cells (FIG. 4). Hs766T arrested in G2/M at a MMC concentration of 100 nM, PL11 at 100 nM, CAPAN1 at 200 nM and FaDu at 500 nM, whereas control pancreatic cancer cell lines MiaPaCa2, Su86.86 and BxPC3 arrested at MMC concentrations as high as 2 μM. These results further established the hypersensitivity of FA-defective cancer cells to crosslinking agents.

All publications, patents and patent applications disclosed herein are incorporated into this application by reference in their entirety; including U.S. Pat. No. 5,952,190, US Patent Application No. 20030188326 and “Sambrook et al., Molecular Cloning. A Laboratory Manual (volumes I-III) 1989, Cold Spring Harbor Laboratory Press, USA”, “Harlowe and Lane, Antibodies a Laboratory Manual 1988 and 1998, Cold Spring Harbor Laboratory Press, USA” and “Ausubel et al., Current Protocols. 2001, John Wiley and sons, Inc.” provide sections describing methodology for antibody generation and purification, diagnostic platforms, cloning procedures, etc. that may be used in the practice of the instant invention.

1. A method of diagnosing or determining if a patient has cancer or is at increased risk of cancer, the method comprising testing a FANC gene for the presence of a cancer-associated coding change, wherein said presence of one or more cancer-associated coding changes is indicative of cancer or an increased risk of cancer in said patient.

2. The method according to claim 1, wherein said cancer is pancreatic cancer.

3. The method according to claim 1, wherein said cancer-associated coding change in the FANCC gene is selected from the group consisting of: mutations of the FANCA gene listed in Table 2, mutations of the FANCC gene listed in Table 3, mutations of the FANCD2 gene listed in Table 4, mutations of the FANCE gene listed in Table 5, mutations of the FANCF gene listed in Table 6, mutations of the FANCG gene listed in Table 7.

4. A method of determining if a patient has cancer, or is at increased risk of developing cancer comprising the steps of: (a) providing a DNA sample from said patient; (b) amplifying the FANC gene from said patient with the FANC gene-specific polynucleotide primers; (c) sequencing the amplified FANCC gene; and (d) comparing the FANC gene sequence from said patient to a reference FANC gene sequence, where a discrepancy between the two gene sequences indicates the presence of a cancer-associated coding change; wherein the presence of one or more cancer-associated coding changes indicates said patient has cancer or is at an increased risk of developing cancer.

5. The method according to claim 4, wherein said cancer-associated coding change in the FANCC gene is selected from the group consisting of: mutations of the FANCA gene listed in Table 2, mutations of the FANCC gene listed in Table 3, mutations of the FANCD2 gene listed in Table 4, mutations of the FANCE gene listed in Table 5, mutations of the FANCF gene listed in Table 6, mutations of the FANCG gene listed in Table 7.

6. The methods according to claim 4, wherein said cancer is pancreatic cancer.

7. A method of treating a patient having cancer or having a risk of developing cancer who has one or more cancer-associated coding changes in the FANC genes comprising the step of administering a therapeutically effective amount of a chemotherapeutic DNA cross-linking agent.

8. The method according to claim 7, wherein said cancer-associated coding change in the FANCC gene is selected from the group consisting of: mutations of the FANCA gene listed in Table 2, mutations of the FANCC gene listed in Table 3, mutations of the FANCD2 gene listed in Table 4, mutations of the FANCE gene listed in Table 5, mutations of the FANCF gene listed in Table 6, mutations of the FANCG gene listed in Table 7.

9. The methods according to claim 7, wherein the said cancer is pancreatic cancer.

10. The methods according to claim 7, wherein the therapeutically effective amount of a chemotherapeutic DNA cross-linking agent is a low dose of agent.

11. The method according to claim 10, wherein the low dose of agent is a daily dose at one-twentieth to one-fifteenth the standard dose.

12. The method according to claim 7, wherein said chemotherapeutic cross-linking agent is mitomycin C.

13. The method according to claim 7, wherein said chemotherapeutic cross-linking agent is cisplatin.

14. A method for screening for a cancer therapeutic, the method comprising the steps of: (a) providing one or more cells containing one or more cancer associated coding changes in the FANC genes; (b) growing said cells in the presence of a potential cancer therapeutic; and (c) determining the rate of growth of said cells in the presence of said potential cancer therapeutic relative to the rate of growth of equivalent cells grown in the absence of said potential cancer therapeutic; wherein a reduced rate of growth of said cells in the presence of said potential cancer therapeutic, relative to the rate of growth of equivalent cells grown in the absence of said potential cancer therapeutic, indicates that the potential cancer then is a cancer therapeutic.

15. The method of claim 14, wherein said cells containing one or more cancer associated coding changes in the FANC genes are distributed in a array.

16. A kit for detecting cancer-associated coding changes in the FANC gene, comprising a polynucleotide primer pair specific for the FANC gene, a reference FANC gene sequence and packaging materials therefore.

17. A microarray containing one or more nucleic acid sequences from one or more FANC genes with cancer-associated coding changes.

18. The microarray of claim 17, wherein said cancer-associated coding change in the FANCC gene is selected from the group consisting of: mutations of the FANCA gene listed in Table 2, mutations of the FANCC gene listed in Table 3, mutations of the FANCD2 gene listed in Table 4, mutations of the FANCE gene listed in Table 5, mutations of the FANCF gene listed in Table 6, mutations of the FANCG gene listed in Table 7.

19. The microarray of claim 18, wherein said cancer-associated coding change in the FANCG gene is selected from the group consisting of: E105ter with loss of heterozygosity; and S7F with loss of heterozygosity.

20. A method for screening for a cancer therapeutic, the method comprising the steps of: (a) providing one or more cells containing one or more cancer associated coding changes in the FANCC, FANCG or BRCA2 genes; (b) growing said cells in the presence of a potential cancer therapeutic; and (c) determining the rate of growth of said cells in the presence of said potential cancer therapeutic relative to the rate of growth of equivalent cells grown in the absence of said potential cancer therapeutic; wherein a reduced rate of growth of said cells in the presence of said potential cancer therapeutic, relative to the rate of growth of equivalent cells grown in the absence of said potential cancer therapeutic, indicates that the potential cancer then is a cancer therapeutic.

21-31. (canceled)
